(19) United States
(12) Patent Application Publication
(10) Pub. No.: US 2020/0372980 A1
(43) Pub. Date: Nov. 26, 2020

(54) METHOD AND SYSTEM FOR ENSURING DATA INTEGRITY

(71) Applicant: Medidata Solutions, Inc., New York, NY (US)

(72) Inventors: Glen de Vries, New York, NY (US); Michelle Marlborough, Brooklyn, NY (US)

(21) Appl. No.: 16/991,915

(22) Filed: Aug. 12, 2020

Related U.S. Application Data
(63) Continuation-in-part of application No. 14/140,734, filed on Dec. 26, 2013, now abandoned.

(51) Int. Cl. G16H 10/20; G06F 11/10; G16H 40/20; G06Q 50/26

(52) U.S. Cl. CPC G16H 10/20; G06Q 50/26; G16H 40/20; G06F 11/10

(57) ABSTRACT

A method for ensuring the integrity of data includes receiving audits from an eClinical system, generating a data stream and a first audit stream, generating a first hash number by applying a hash algorithm to the first audit stream, transmitting the data stream and first audit stream to a data provider, and transmitting the first hash number to an error checker. The data provider provides to the error checker a second audit stream, and the error checker generates a second hash number based on the second audit stream and compares the first hash number to the second hash number. A system for ensuring the integrity of data is also described and claimed.

### CLAIM OF PRIORITY

This application is a continuation-in-part of and claims priority from U.S. application Ser. No. 14/140,734, filed Dec. 26, 2013, the entirety of which is incorporated herein by reference.

### FIELD OF THE INVENTION

The current disclosure relates to ensuring the integrity of data transmitted from one location to another, e.g., to ensure that the transmission did not introduce any errors in the data or that the data was not otherwise tampered with.

### BACKGROUND

Data transmitted from one location to another may need to be verified to ensure that the transmission did not introduce any errors in the data. One example of such data transmission occurs in clinical studies, also known as clinical trials. These studies are typically conducted to evaluate the safety and efficacy of medicines, medical devices, or other medical treatments by monitoring and studying their effects on groups of people. Using clinical studies, doctors and researchers may find new and better ways to prevent, detect, diagnose, or treat diseases. A clinical study is often sponsored by a drug manufacturer (sometimes called the “sponsor”) and may be carried out by a contract research organization (“CRO”), and may involve numerous entities such as hospitals, doctors (principal investigators), nurses, patients, and site monitors. Findings or results from these clinical studies may then be sent by the sponsor to regulatory agencies such as the United States Food and Drug Administration (“FDA”) or the European Medicines Agency (“EMA”).

During the course of a clinical study, a large amount of clinical data and information may be gathered at various investigator sites, such as hospitals and clinics, by personnel such as doctors, patients, nurses, and technicians. These data may be inputted into a system where they may be recorded and stored. These data may then be transmitted by the sites to, for example, CROs, sponsors, and/or regulatory agencies. In some cases, an investigator site may transmit the data to a CRO, which may in turn forward that data to a sponsor that may finally submit the data to a regulatory agency, such as the FDA or EMA.

### BRIEF DESCRIPTION OF THE DRAWINGS

FIGS. 1 and 2 are block diagrams of systems that use hash numbers to ensure the integrity of data, according to embodiments of the present invention;

FIG. 3 is a block diagram of another system that uses hash numbers to ensure the integrity of data, according to another embodiment of the present invention;

FIG. 4 illustrates how data may be changed between the time of a clinical study and a submission to a regulatory agency, according to an embodiment of the present invention;

FIGS. 5A-5D show examples of appended data streams, according to embodiments of the present invention; and

FIG. 6 is a flowchart illustrating how hash numbers may ensure the integrity of clinical data, according to an embodiment of the present invention.

Where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of embodiments of the invention. However, it will be understood by those of ordinary skill in the art that the embodiments of the present invention may be practiced without these specific details. In other instances, well-known methods, procedures, components, and circuits have not been described in detail so as not to obscure the present invention. The present invention is not intended to be limited to any particular operating system, software application, or market. Additionally, any examples of particular software applications or markets used herein are included for illustration purposes and are not intended to be limiting.

With the advent of computer and network technologies, data may be collected using electronic means during the course of a clinical study. Electronic data collection may present challenges in ensuring that the data transmitted from one organization to another are accurate and valid. It may be a challenge to keep track of updates or changes made to the clinical data over the course of a clinical study. It may also be difficult to trace back to such updates and changes that may be made at a given time during the clinical study.

A regulatory agency does not generally have the ability to accurately and rapidly assess whether the data that it receives from a life sciences company, such as a drug sponsor, for regulatory purposes have been altered in any way. For example, the FDA may receive, at the end of a clinical study, a copy of the data from the sponsor, which certifies that the data are as accurate as the data collected at the source. However, even though current clinical applications may include auditing capabilities, it may be difficult (if not impossible) for the FDA to fully verify quickly whether the data have been altered, either inadvertently or intentionally, by the sponsor or someone else in the data transmission chain. Thus, a regulatory agency would like to ensure there has not been any data tampering, corruption, or change between the time the clinical data were collected and the time when it receives the data. Regulatory agencies also often require site personnel to certify at the end of a study or when a patient completes his or her participation in a study that the data transmitted from the site to the sponsor are the same as the data that were entered by site personnel into various eClinical systems during the course of the study, i.e., that the site has been in control of its data throughout the process of data capture, cleaning, and submission to the agency.

A system for ensuring that clinical data submitted to a regulatory agency are accurate and valid has been developed. This system may collect data from a clinical study and then may apply an algorithm to the stream of collected data to generate a single number representative of the collected data stream. The collected data may then be transmitted to another entity, such as a sponsor, which then prepares a submission to the regulatory agency in support of regulatory approval of the item being studied. The submission may include the sponsor's version of the collected data. The regulatory agency may then verify that the data from the sponsor are the same as the data collected during the study by applying the same algorithm to the sponsor's data and comparing the representative number from that algorithm to the representative number previously generated. If the representative numbers differ, the regulatory agency knows that the data from the sponsor are not the same as the data transmitted to the sponsor. The system may also be used by site personnel to verify that the data the site generated are being transmitted to the sponsor and the regulatory agency.

The algorithm applied to the data streams may be a hashing algorithm and the single number generated that is representative of the data stream may be a hash number. Generally, hashing is a transformation of a set of data into, for example, a value of a pre-determined length that reflects that set of data. A set of data that may be hashed includes, for example, a string or a page of alphanumerical characters, an entire electronic data file, and an electronic form with multiple fields. Hashing algorithms that may be used in conjunction with this system may include, but are not limited to, the MD5 algorithm, the MD6 algorithm, and customized hashing programs. Hashing the data stream allows for much more rapid verification of data integrity than comparing the two sets of data line-by-line or field-by-field, which may be time consuming, cost prohibitive, cumbersome, and error prone.

A further feature of the present invention is the ability to take into account all of the information related to a set of clinical data, which information may be represented by a set of audits. As used herein, an audit may be a record of a transaction occurring at one or more clinical data sources. An audit may include clinical data, operational data, or both, generated as a result of the transaction executed at the data source. Clinical data may include height, weight, blood tests, blood pressure, activity metrics, glucose levels, ECG data, and other pharmacokinetic and pharmacovigilance data. Operational data may include time stamps, vector stamps, and, more broadly, causality-determining markers associated with an executed transaction. Operational data may also include data regarding what action was taken, who took the action, the identity of a device used to take the action (e.g., record some data), on whose behalf the action was taken, when the action was taken, what was changed from a previous state, the reason for the change, and what other audits may be related to it (e.g., identified by transaction ID), along with other information. (An “action” as used herein may include recording, calculating, converting, or transmitting data, and may be a subset of or coextensive with a transaction.) Audits may ultimately provide a permanent and indelible record, in keeping with the regulatory requirements that govern many clinical study systems. Thus, embodiments of the present invention involve hashing audit streams rather than just clinical data streams.

The system is not limited to ensuring the integrity of data submitted to a regulatory agency from a sponsor in the context of a clinical study, but may encompass situations in which the integrity of data that are transmitted to multiple entities needs to be ensured.

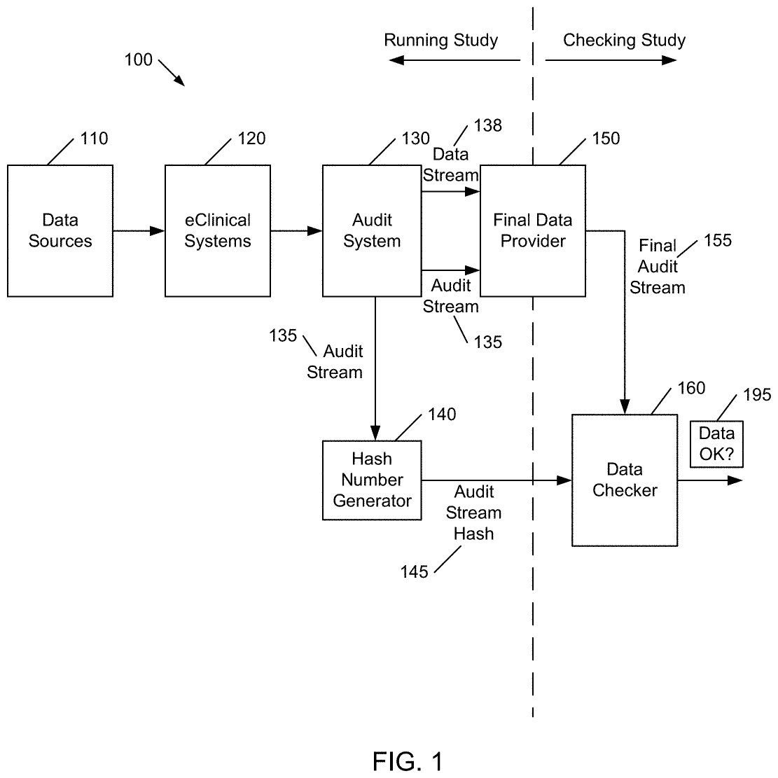

Reference is now made to FIG. 1, which is a block diagram of system 100 that uses hash numbers to ensure the integrity of data. FIG. 1 is divided into two main parts—one in which the study is running or operating (“running study”) and one in which some entity may check the study and the integrity of the study data (“checking study”). System 100 may include data sources 110 providing clinical study data to eClinical systems 120, which in turn may provide audits to audit system 130. Audit system 130 may provide data stream 138 and audit stream 135 to final data provider 150, and at the same time may hash the audit stream using hash number generator 140 to produce audit stream hash 145 to be provided to data checker 160, which may check the integrity of the data. Final data provider 150 may provide a final audit stream 155 to data checker 160, which may re-hash final audit stream 155 and determine at 195 whether the data and audits from final data provider 150 are trustworthy.

Data sources 110 may include sources that provide, for example, electronic data, medical image data, medical instrument data, blood test results, pharmacy records, various clinical analysis data, and scanned paper document data, just to name some of the types of sources. More specific examples of such data are patient x-ray images or CT scan images from an imager, a patient's body temperature measured from a digital thermometer, various blood measurements obtained from a digital blood analysis machine, a pharmacy record obtained from a pharmaceutical dispensing management system, and a physician's analysis scanned from a paper-based document. Besides patient-related data, there may be other data related to a clinical study, such as operational data, summary data, and payment data.

In a clinical study, such data may come from patients, principal investigators, nurses, technicians, and clinical research associates (CRAs), among others. eClinical systems 120 may include electronic data capture (EDC) systems, electronic medical records (EMR) systems, electronic health records (EHR) systems, eCRF (electronic case report form) systems, clinical data management (CDM) systems, randomization systems, coding systems, health or activity tracking devices, and ECG and glucose monitors, among other electronic and/or web-based systems used for the capture of clinical trial data.

Audit system 130 collects audits from the various eClinical systems and, because audits may be used as a permanent record of the clinical study, may format the audits in accordance with rules provided by the data checker. In one embodiment of the present invention, audit system 130 may be operated by a third party (that is, a party that is different from final data provider 150 and data checker 160) that collects and assembles the audit stream and then transmits it to data provider 150 and to data checker 160, along with audit stream hash 145. The third party may be considered to be a “trusted” or “independent” third party by data checker 160.

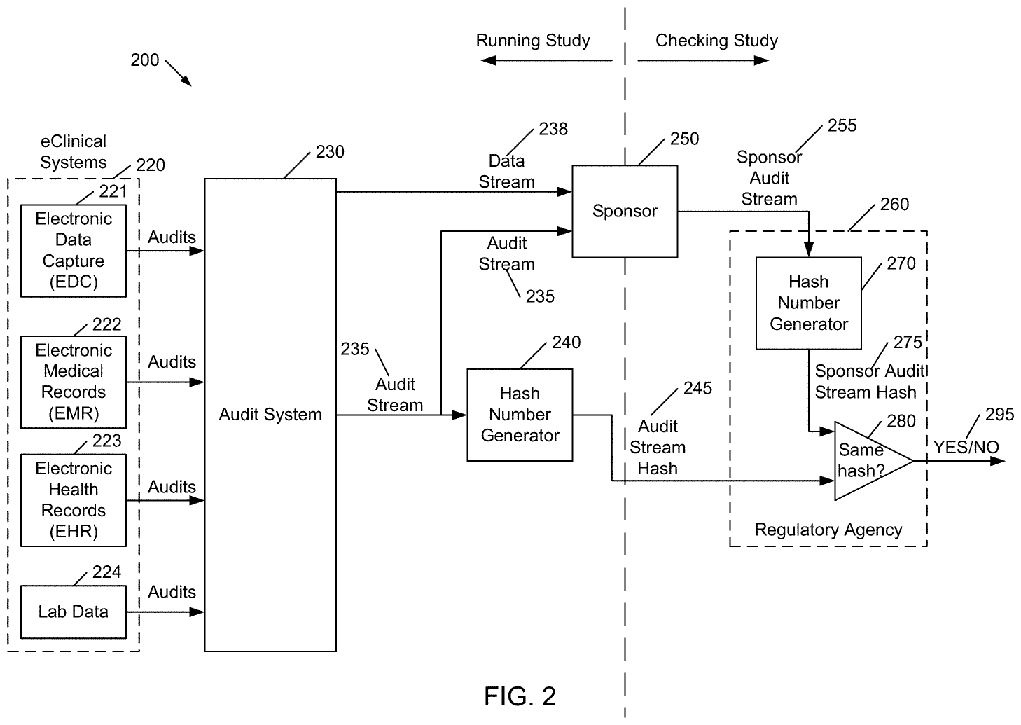

Reference is now made to FIG. 2, which is a block diagram of system 200 that uses hash numbers to ensure the integrity of clinical data, and is generally a more specific embodiment of FIG. 1. eClinical systems 220 is shown as explicitly including EDC module 221, EMR module 222, EHR module 223, and lab data module 224. Audit system 230 operates the same way as audit system 130. Sponsor 250 is an example of final data provider 150 in FIG. 1, and regulatory agency 260 is an example of data checker 160 in FIG. 1.

Each of the eClinical systems may produce audits and transmit them to audit system 230. The audits may be appended by audit system 230 into audit stream 235, which may then be input to hash number generator 240, producing audit stream hash 245. Audit system 230 may then provide audit stream 235 to sponsor 250, possibly along with data stream 238. Audit system 230 may provide audit stream hash 245 to regulatory agency 260. Sponsor 250 may provide a package to regulatory agency 260, so as to meet the requirements of the regulatory agency with respect to, for example, approval for a drug based on the clinical study. This package may include sponsor audit stream 255 (and may also include a sponsor data stream (not pictured)). Regulatory agency 260 then may review the package submitted by the sponsor. If the regulatory agency wants to quickly determine whether sponsor audit stream 255 is the same as audit stream 235 that was actually produced during the clinical study, regulatory agency 260 may hash sponsor audit stream 255 using hash number generator 270 to generate sponsor audit stream hash 275 and may then use comparator 280 to compare audit stream hash 245 and sponsor audit stream hash 275. Discrepancies in the hash numbers indicate differences in the audit streams, which may indicate errors in the data or that at least one part of the data from the study has been inadvertently or intentionally changed or tampered with.

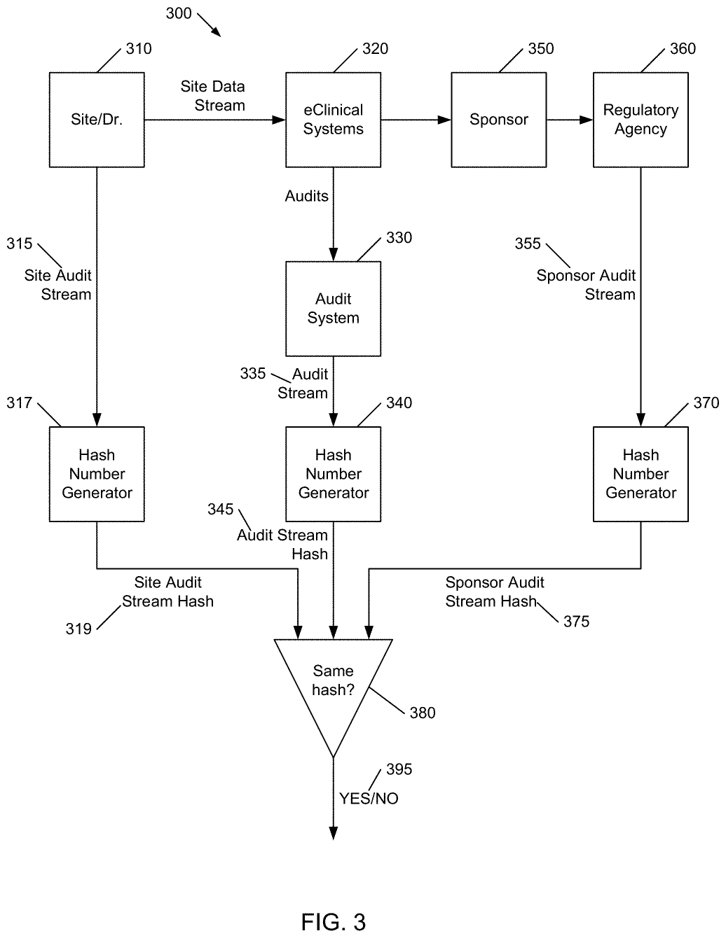

In a manner similar to the way the regulatory agency may verify data integrity by using the hashing techniques of the present invention, so too may site personnel, such as a doctor, principal investigator, or other health care professional who may have input the data, use such hashing techniques, as illustrated in FIG. 3. As with system 100 in FIG. 1, system 300 includes site/Dr. 310 as a data source, which may provide a data stream to eClinical systems 320. Sponsor 350 may take the data (and/or audits) from eClinical systems 320 to prepare its submission to regulatory agency 360. As in FIG. 1, eClinical systems 320 may provide audits to audit system 330, which may generate audit stream 335. Audit stream 335 may be hashed using hash generator 340 to generate audit stream hash 345. As before, audit system 330 may be operated by a trusted or independent third party.

As was also discussed with respect to FIG. 2, regulatory agency 360 may verify that audit stream 355 it was provided by the sponsor is the same as audit stream 335 generated by audit system 330. This may be accomplished by hashing sponsor audit stream 355 using hash number generator 370 and comparing sponsor audit stream hash 375 to audit stream hash 345 using comparator 380. Output 395 will inform regulatory agency 360 if the hash numbers are the same are different. In FIG. 3, site/Dr. 310 may verify that the audit stream 315 it generated is the same as audit stream 335 generated by audit system 330. This may be accomplished by hashing site audit stream 315 using hash number generator 317 and comparing site audit stream hash 319 to audit stream hash 345 using comparator 380. Output 395 will inform site/Dr. 310 if the hash numbers are the same are different. This embodiment may be useful if the site and/or the site personnel are required to certify that the data that the site generated are the same as the data transmitted to the sponsor and to the regulatory agency.

The blocks shown in FIGS. 1-3 are examples of modules that may comprise systems 100, 200, and 300, and do not limit the blocks or modules that may be part of or connected to or associated with these systems. For example, not only may a regulatory agency be a data checker, but any entity downstream from where the data are collected may be a data checker, including a provider, a CRO, a patient, a sponsor, or another third party. The sources of data are not limited to just patients, but may include providers, CROs, sponsors, and other third parties. And the final data providers are not limited to just sponsors, but may include providers, CROs, and other third parties. Thus, a CRO may be a final data provider and a sponsor may be a data checker. In addition, the audit system is not limited to an actual machine or system—it may be a format that eClinical systems adopt for audits so that the audit stream transmitted to the data checker is trustworthy and intact. Moreover, while the hash number generators are shown as distinct blocks, the audit system, site/Dr., regulatory agency, and/or data checker may comprise and/or control the respective hash number generators. And while audit streams are shown as inputs to hash number generators, a data stream may be hashed instead of or in addition to an audit stream.

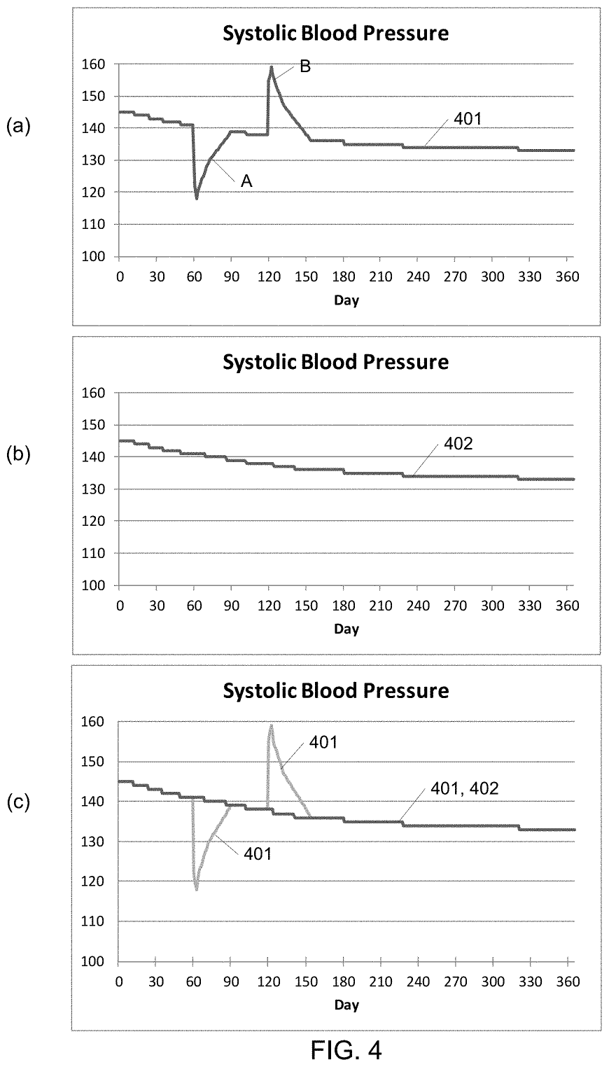

The benefit of the type of hashing used in the present invention is that if there is any tampering with the data and/or audits, a single hashing of the altered audit stream will uncover such tampering because it will differ from the audit stream hash. That situation is demonstrated in FIG. 4, which illustrates how data may be changed between the time of the trial and the submission to the regulatory agency. Graph (a) is an exemplary graph of daily systolic blood pressure (SBP) readings of a patient P during a clinical trial that lasts 365 days. Trace 401 shows the actual SBP readings for the year, with episodes A and B in which the SBP readings markedly changed over the course of a number of days. These changes may be due, for example, to adverse events during the study. Referring back to FIG. 2 for simplicity of explanation, trace 401 may be included in audit stream 235, and audit system 230 may provide audit stream hash 245 to regulatory agency 260.

Sponsor 250 may receive audit stream 235 and notice that the SBP readings for patient P are not favorable. Sponsor 250 may then attempt to modify the SBP readings of patient P to follow trace 402, shown in graph (b), that removes episodes A and B. (Graph (c) shows both traces superimposed.) Trace 402 would then be included in sponsor audit stream 255. Sponsor 250 may then provide sponsor audit stream 255 to regulatory agency 260.

Upon receiving sponsor audit stream 255, regulatory agency 260 may then perform a hash of sponsor audit stream 255 and compare sponsor audit stream hash 275 to audit stream hash 245 and determine at 295 that the data were actually changed.

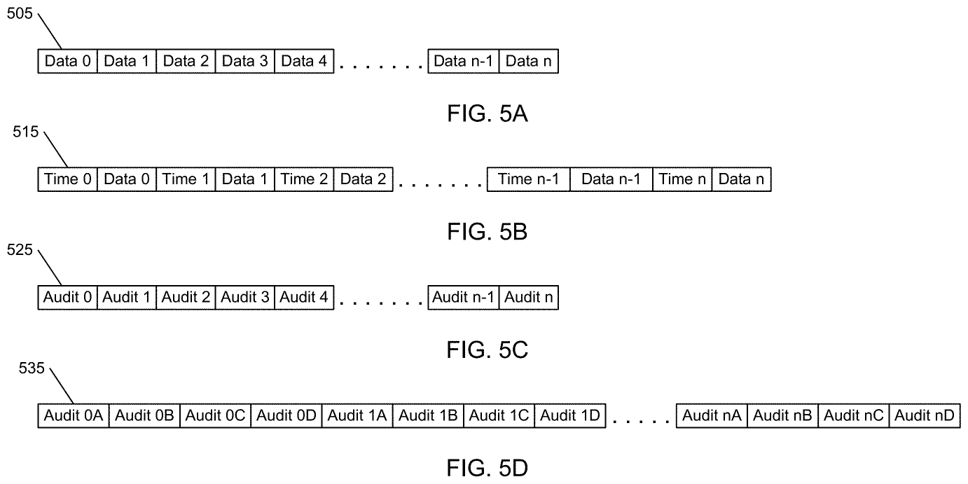

Examples of appended data streams are shown in FIGS. 5A-5D. In FIG. 5A, data stream 505 may include a single piece or type of data, e.g., systolic blood pressure, for a patient for each day of a clinical study of length “n,” as in FIG. 4. An embodiment of the present invention then may perform a hash of data stream 505. Alternatively, each data block in data stream 505 may represent more than a single piece of data, e.g., a patient's full data record for a specific day, which may be recorded on an eCRF (electronic case report form). Data stream 515 in FIG. 5B is a variation of data stream 505. Data stream 515 may also include time stamps for each piece or type of data. Performing a hash on this data may make the hash number more secure because it involves more data. Data stream 525 in FIG. 5C is another variation of data stream 505. Data stream 525 may include the audits for the study appended to each other (and thus may be more properly called an audit stream). The audits may be related to a single piece or type of data, e.g., SBP, or to groups of data, e.g., eCRF. The audit includes the data itself plus the other information (e.g., who, what, where, when, and why) associated with the data. FIG. 5D shows another variation of a data stream or an audit stream. Each block of data may be an audit for a specific day for a specific eClinical system, e.g., modules 221-224 in FIG. 2. Thus audit 0A may be EDC data from day 0, audit 0B may be EMR data from day 0, etc., audit 1A may be EDC data from day 1, etc. The data stream and audit streams in FIGS. 5A-5D are examples of how pieces of data or blocks of audits may be appended by an audit system.

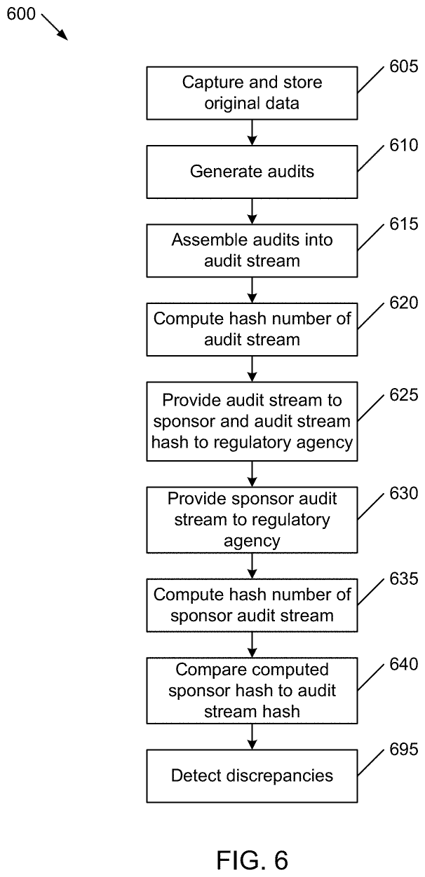

FIG. 6 is a flowchart 600 illustrating the operation of system 200 in FIG. 2 (and many of the operations may be used in systems 100 and 300). Hashing may be performed on an entire data stream or audit stream, such as those shown in FIGS. 5A-5D. In operation 605, data may be captured, e.g., by eClinical systems 221-224, and stored. eClinical systems 221-224 may generate audits based on the data, in operation 610. In operation 615, audit system 230 may assemble the audits into an audit stream, such as audit stream 235 (or audit streams 525 or 535). In operation 620, hash number generator 240 may compute audit stream hash 245 for audit stream 235. In operation 625, audit system 230 may provide audit stream 235 (and/or data stream 238) to sponsor 250 and audit stream hash 245 to regulatory agency 260. In operation 630, sponsor 250 may provide sponsor audit stream 255 to regulatory agency 260.

Next, in operation 635, regulatory agency 260 may compute the hash number of sponsor audit stream 255 using hash number generator 270 and compare that hash number to audit stream hash 245 in operation 640. If there are any discrepancies detected in operation 695, then the regulatory agency knows that the audit stream has been altered or that there are errors in the data.

Besides the operations shown in FIG. 6, other operations or series of operations may be used to verify the integrity of the data generated in a clinical environment. Moreover, the actual order of the operations in the flowchart may not be critical. For example, more than one hash number may be produced for a study or for different sets of data. Hash numbers may be generated for pieces or types of data, e.g., at the end of every day, for systolic blood pressure over a period of time or over the course of the study, for partial or completed eCRFs, for a specific patient, for a site, etc. In fact, any block of data that may need to be verified later may be hashed. The hashing may be of data streams, or audit streams, or both.

Data and audits from a clinical study are only one example of how the invention may be used—other scenarios exist in which clinical data may need to be verified. One scenario is ensuring quality in pharmaceutical manufacturing facilities, where certain data, such as temperature, pH, etc., may need to be collected for each bottle, and the manufacturing facility keeps audit records that may be checked later by an assurance agency. Another scenario is airline maintenance, where records may need to be kept to ensure ongoing quality and to determine whether anything wrong occurred in the case of an investigation. More generally, the present invention may be used in industries and scenarios in which there is a requirement (whether legal or not) to keep data and records.

In addition, the present invention may also be used to operate on data that do not comprise the complete data stream from a study. Hash numbers of pieces of data or of cumulative data may be transmitted to the data checker, for example, during a study, and then the hash number may be updated at a different time, for example, the next day. Such updates may occur regularly, at consistent intervals, or periodically, at varying intervals. Because the updated data or audit stream may include more bits, the hash number becomes stronger. The data and audit streams may also have associated time stamps, further strengthening the resulting hash numbers.

The present invention may keep track of and record every data entry event, including adding, modifying, and deleting data. The audit stream includes the data plus all the details about the data, such as operational data and metadata. By assembling the audits into a cumulative audit stream and then computing a hash number based on the cumulative audit stream, the present invention allows a data checker to rapidly verify the integrity of clinical data it receives. In addition, the present invention accumulates audits from a number of clinical applications (e.g., eClinical systems) and hashes the resulting cumulative stream, whereas prior auditing capabilities were generally limited to that specific application, with no comprehensive auditing capability.

Aspects of the present invention may be embodied in the form of a system, a computer program product, or a method. Similarly, aspects of the present invention may be embodied as hardware, software or a combination of both. Aspects of the present invention may be embodied as a computer program product saved on one or more computer-readable media in the form of computer-readable program code embodied thereon.

For example, the computer-readable medium may be a computer-readable storage medium. A computer-readable storage medium may be, for example, an electronic, optical, magnetic, electromagnetic, infrared, or semiconductor system, apparatus, or device, or any combination thereof.

Computer program code in embodiments of the present invention may be written in any suitable programming language, including C, Objective-C, C # (c-sharp or .NET), JavaScript, Ruby, and others. The program code may execute on a single computer or on a plurality of computers. The computer may include a processing unit in communication with a computer-usable medium, wherein the computer-usable medium contains a set of instructions, and wherein the processing unit is designed to carry out the set of instructions.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A method for ensuring the integrity of data, comprising:
receiving audits from an eClinical system;
generating a data stream and a first audit stream;
generating a first hash number by applying a hash algorithm to the first audit stream;
transmitting the data stream and first audit stream to a data provider; and
transmitting the first hash number to an error checker,
wherein:
the data provider provides to the error checker a second audit stream based on the first audit stream; and
the error checker generates a second hash number based on the second audit stream and compares the first hash number to the second hash number to detect errors in the data.

2. A system for ensuring the integrity of data, comprising:
an audit system configured to receive data from an eClinical system, generate a data stream and a first audit stream, and transmit the data stream and first audit stream to a data provider; and
a hash number generator configured to generate a first hash number of the first audit stream and transmit the first hash number to an error checker,
wherein:
the data provider transmits to the error checker a second audit stream based on the first audit stream; and
the error checker generates a second hash number based on the second audit stream and compares the first hash number to the second hash number to detect errors in the data.
